(19) United States
(12) Patent Application Publication
(10) Pub. No.: US 2022/0081660 A1
(43) Pub. Date: Mar. 17, 2022

(54) Shape-Complementing, Porosity-Matching Perfusion Bioreactor System for Engineering Geometrically Complex Tissue Grafts

(71) Applicant: CARNEGIE MELLON UNIVERSITY, Pittsburgh, PA (US)

(72) Inventors: Xi Ren, Pittsburgh, PA (US); Barbie Clara Varghese, Pittsburgh, PA (US); Ananya Sushanta Kar, Pittsburgh, PA (US); Julia Eve Napolitano, Pittsburgh, PA (US); Yunhui Xing, Pittsburgh, PA (US)

(73) Assignee: CARNEGIE MELLON UNIVERSITY, Pittsburgh, PA (US)

(21) Appl. No.: 17/478,202

(22) Filed: Sep. 17, 2021

Related U.S. Application Data
(60) Provisional application No. 63/079,497, filed on Sep. 17, 2020.

(51) Int. Cl. C12M 3/00; A61F 2/28; A61F 2/30; C12M 1/12

(52) U.S. Cl. CPC C12M 21/08; A61F 2/2875; A61F 2002/30985; C12M 25/14; A61F 2002/2817; A61F 2/3094

(57) ABSTRACT

A perfusion bioreactor system has an inner chamber and scaffold with matching porosities to equalize fluid flow through a bioreactor. The scaffold can be fabricated using additive manufacturing or other fabrication techniques to match the geometrical shape of a defect, such as a facial bone anomaly. The inner chamber is fabricated in a similar manner and has an inner cavity matching the shape of the scaffold to create a unified structure when assembled together with the scaffold. By matching the shapes of the scaffold and inner chamber, free space is eliminated within the interior volume of the bioreactor. Stem cells can be flowed through the bioreactor and attached to the scaffold, which are then cultured to grow a tissue graft.

### CROSS-REFERENCE TO RELATED APPLICATIONS

This application claims the benefit under 35 U.S.C. § 119 of U.S. Provisional Application Ser. No. 63/079,497, filed Sep. 17, 2020, which is incorporated herein by reference.

### STATEMENT REGARDING FEDERALLY SPONSORED RESEARCH

Not applicable.

### BACKGROUND OF THE INVENTION

The present disclosure is related generally to bioreactors. More specifically, the disclosure is related to a perfusion bioreactor system used to create geometrically complex bone grafts.

Autologous bone grafting is considered the current gold standard for reconstructive and cosmetic surgical interventions. While the innate osteogenic, osteoconductive, and osteoinductive properties of autologous bone grafts offer a certain convenience, they are limited by problems with donor site morbidity, tissue availability, and the inability to sculpt complex, defect-specific geometries from the donor bone. The success of bone reconstructions, especially in the craniofacial region, is distinctly dictated by the geometric compatibility between the graft and the defect being treated. Hence, bone grafts with the greatest functionality would possess both a diverse cocktail of cells and biomaterials to boost regeneration, as well as defect-specific geometries.

Tissue scaffolds have been used to create tissue grafts, where cells attach to the scaffold and stimulate the formation of new tissue. In many cases, a porous biomaterial scaffold may be generated and implanted to stimulate cell growth in vivo. While such a scaffold may fix the defect and restore functionality, it relies on cells present around the defect to infiltrate the porous scaffold and promote bone regeneration at the site of the defect. However, relying on cell infiltration alone to facilitate bone regeneration becomes less ideal when the scaffolds are larger and possess critical geometries. For larger defects or defects with complex geometries, it is beneficial to cellularize the scaffold in vitro prior to implantation, giving the grafts a better chance at regenerating bone in vivo.

The scaffold can be created in a variety of shapes to match the shape of the defect. Additive manufacturing has emerged as a promising strategy for manufacturing such ‘designer scaffolds’ with intricate geometries. However, the ultimate challenge lies in meeting the oxygen and nutrient demands of culturing such physiologically relevant bone grafts in industrial or clinical labs prior to implantation. Traditional perfusion bioreactors were conceived as a strategy to enhance fluid flow and mass transport across such scaffolds. Despite a bioreactors general performance for culturing cells, in use with scaffolds of varying sizes and geometries they are severely restricted by the lack of uniform perfusion across the scaffold, which is critical to the formation of robust bone tissue with uniform mechanical properties and morphology across the desired scaffold. The lack of uniform perfusion can become more pronounced as the shape and geometry of the scaffold becomes more complex. Therefore, it would be advantageous to create a system that facilitates the production of large, geometrically complex bone substitutes.

### BRIEF SUMMARY

According to embodiments of the present disclosure is a perfusion bioreactor system that houses a porous inner chamber, which may be implemented as a multi-part component and is customized to complement the geometry and porosity of the desired scaffold. Coordinating the geometry and porosity in this manner will yield an inner compartment within the bioreactor free from preferential fluid flow, achieving uniform perfusion across the scaffold irrespective of size and geometry. The resulting bioreactor has the potential to facilitate the uniform delivery of cells, medium, and biomaterials to enable homogeneous tissue growth in personalized bone grafts.

Digitized images of defects can be used to design and manufacture the desired personalized scaffold and corresponding inner chamber. Additive manufacturing and other fabrication techniques can be used to create the scaffold and inner chamber from the digital design. When used in the bioreactor, the scaffold can be coated with an extracellular matrix (ECM) biomaterial, such as collagen, to enhance cell attachment. Additionally, the inner chamber can be coated with an anti-fouling agent to prevent superfluous cell attachment, confining the attachment of cells to the scaffold. The coated parts of the bioreactor can be assembled in a biosafety cabinet to maintain sterility. Stem cells can be dynamically seeded in the bioreactor and perfused with media to support cell proliferation and differentiation into desired lineages. In one example, mesenchymal stem cells (MSCs) can be differentiated into osteoblasts for bone tissue engineering using the bioreactor system of the present disclosure.

### BRIEF DESCRIPTION OF THE SEVERAL VIEWS OF THE DRAWINGS

FIG. 1 depicts the components of a perfusion bioreactor.

FIG. 2 shows a tetrahedron mesh of a bioreactor used for fluid simulation.

FIG. 3 is a 3D printed scaffold, with the inner and outer chambers shown.

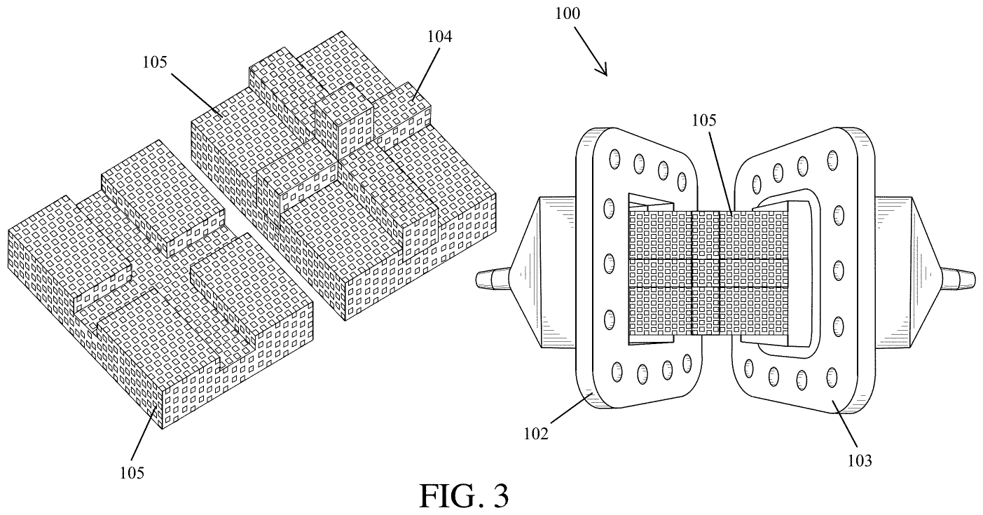

FIGS. 4A-4D show various configurations of a system incorporating a perfusion bioreactor.

FIG. 5 depicts fluid flow across the bioreactor using an inner chamber compared to fluid flow when the inner chamber is omitted.

FIG. 6 are confocal images of hMSC seeded onto a scaffold.

FIG. 7 shows Alizarin red staining confirming the differentiation of hMSCs on a scaffold.

### DETAILED DESCRIPTION

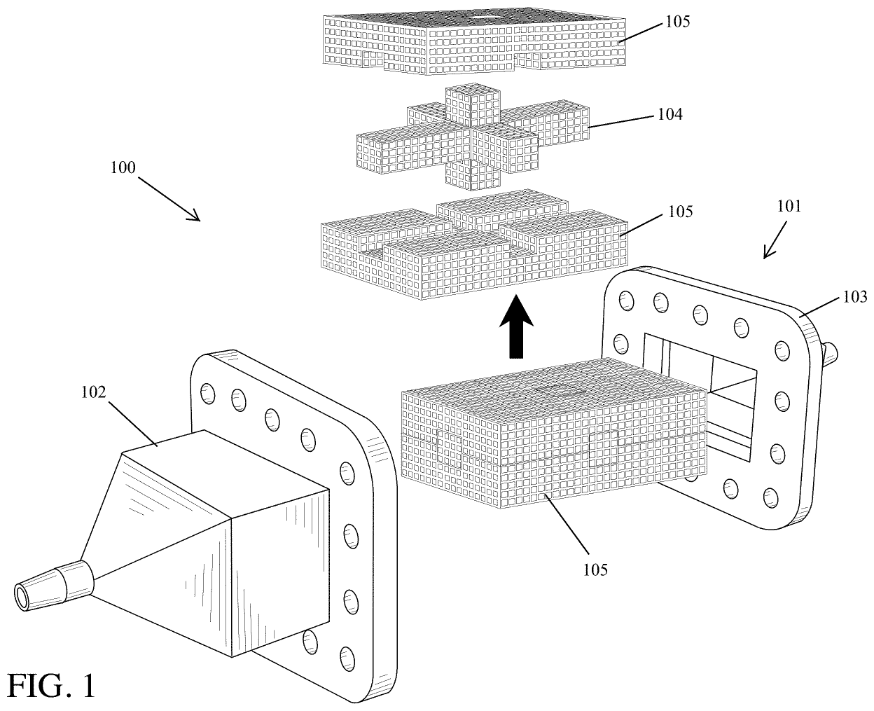

According to embodiments of the disclosure is a perfusion bioreactor system 100, as shown in FIG. 1. The system 100 comprises a bioreactor 101 having an inlet 102 and outlet 103, a scaffold 104, and an inner chamber 105. The inlet 102 and outlet 103 are joined together to form an interior volume of the bioreactor 101. In operation, a fluid is pumped into the inlet 102, passes through the interior volume, and then exits through the outlet 103. The inner chamber 105 may be composed of a plurality of parts and occupies the remainder of the interior volume of the bioreactor 101 that is not otherwise occupied by the scaffold 104. As shown in FIG. 1, the inner chamber 105 comprises two halves that when joined, surround the scaffold 104 and form a structure having a volume that substantially matches the interior volume of the bioreactor 101. Stated differently, the scaffold 104 and inner chamber 105 occupy the interior volume of the bioreactor 100 and eliminate any free space within the interior of the bioreactor 100. When free space is present within the bioreactor 100, fluid flow preferentially follows these areas of reduced resistance, preventing the scaffold 104 from receiving sufficient materials contained within the fluid required for tissue development. However, depending on the shape of the bioreactor 101, the inner chamber 105 may not occupy the space at the ends of the inlet 102 and outlet 103 to allow fluid to freely flow into the interior volume before encountering resistance to flow. In this manner, the ends of the inlet 102 and outlet 103 act as manifolds.

Fabrication of Bioreactor Components

The components of the bioreactor 100 can be designed using computer-aided design (CAD) and/or solid modeling software, such as SOLIDWORKS design software. In one example embodiment, the inlet 102 and outlet 103 may comprise commercial off-the-shelf components. In contrast, the scaffold 104 and porous inner chamber 105 are custom designs, with the geometry of the scaffold 104 based on the defect targeted for repair. Imaging tools such as computed tomography (CT) and magnetic resonance imaging (MRI) can be used to obtain a three-dimensional (3D) rendering of the defect which will be repaired using the graft. Boolean tools can be used to generate a custom CAD model of the scaffold 104 and corresponding inner chamber 105 based on the rendering of the defect.

Other 3D design tools can also be used during fabrication of the scaffold 104. For example, a physical prototype of the scaffold 104 can be created then scanned to digitize its shape and geometry. The resulting digital representation of the prototype can then be refined with software editing tools or fabricated without revisions.

During the design phase, the porosity of the scaffold 104 and inner chamber 105 are determined depending on the intended use. In one example, porosity is implemented at 60% with 600 μm cubical pores and 400 μm struts used in the scaffold 104 and inner chamber 105 structures. Alternatively, algorithmic modeling approaches, such as those found in Grasshopper (Rhino 6) and MATLAB software, can be applied to implement more biomimetic porosity patterns. A difference modifier, such as a Boolean tool on the design software, can be used to generate a cavity in the inner chamber 105 to complement the shape of the scaffold 104. Specifically, the inner chamber 105 is designed to have an inner cavity that matches the shape of the scaffold 104 so that when the two components are joined, there is no free space.

Depending on the shape and complexity of the scaffold 104, the inner chamber 105 may be formed as multiple pieces. In the example shown in FIG. 1, the scaffold 104 is a simple geometric shape used for demonstration purposes and the inner chamber 105 can easily match this shape with two pieces. However, if the scaffold 104 has complex features such as a concavity, interior bend, or non-linear feature, the inner chamber 105 may be created as multiple pieces to permit assembly without void spaces between the scaffold 104 and inner chamber 105.

After completing the design of the scaffold 104 and inner chamber 105, computational fluid dynamics (CFD) models can be generated to assess the homogeneity of fluid perfused through the scaffold 104. For example, a solid model of the internal cavity of the bioreactor 101, representing the region of fluid flow, and a model of the scaffold 104 can be generated. The models can be used to generate the velocity fields for fluid flow through the bioreactor 101 on computational fluid dynamics analysis software, such as ANSYS Fluent, Elmer, and COMSOL Multiphysics, among others. In one example, the bioreactor system 100 is meshed separately as four different parts: the inlet 102, outlet 103, inner chamber 105, and scaffold 104.

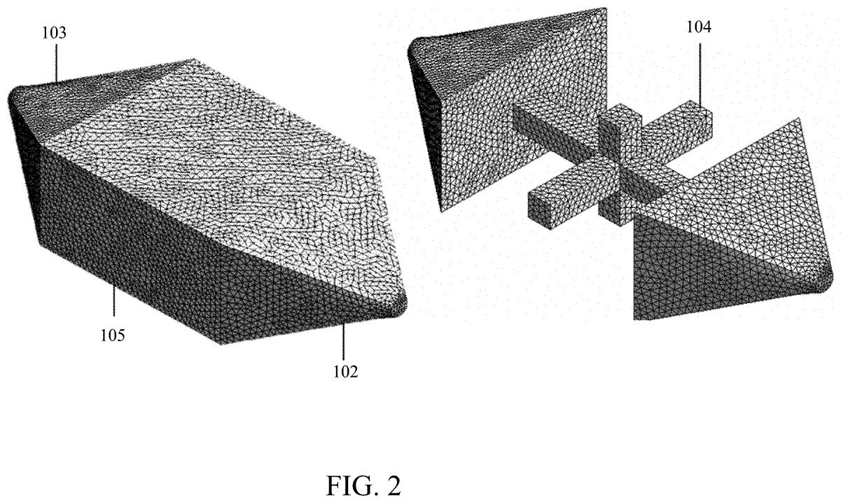

A representation of these models is shown in FIG. 2, with the inlet 102, outlet 103, and inner chamber 105 shown in the image on the left of FIG. 2. The scaffold 104, without the inner chamber 105, is shown in the image on the right of FIG. 2. For modeling purposes, the inner chamber 105 and scaffold 104 are assumed to be porous zones. For example, the porosity of the scaffold 104 is maintained as 94% while the porosity of the inner chamber 105 is kept as either 100% or 94% (same as the scaffold 104). Uniform fluid flow is achieved where the porosity of the inner chamber 105 matches the porosity of the scaffold 104. As previous noted, matching the porosity of the scaffold 104 and inner chamber 105 reduces fluid flow around the scaffold 104 because the inner chamber 105 and scaffold 104 have similar resistance to the flow of fluid. Further assumptions for modeling are laminar flow for an incompressible Newtonian fluid through porous media. The inlet velocity was assumed to be 1 mL/min, and no-slip boundary conditions were imposed at the walls of the bioreactor 101.

The design files of the scaffold 104 and inner chamber 105 can be edited on 3D printing software to incorporate cylindrical supports, resulting in high fidelity, porous prints. The porous parts are printed on printer capable of printing suitable materials for the scaffold 104. In one example, the printer is a M100-405 nm digital light projection (DLP) printer from CADworks3D. The scaffold 104 and inner chamber 105 can be printed using biocompatible materials, such Dental SG, a biocompatible and autoclavable photosensitive resin. Alternatively, the inner chamber 105 may be printed from a less expensive, non-biocompatible material since it is not intended for tissue growth. For post print processing, printed parts can be washed in isopropyl alcohol (IPA) for 1 hour and dried with compressed air. At this stage, supports can be clipped from all parts, UV cured, and autoclaved prior to assembly.

While additive manufacturing provides an efficient platform for fabricating the scaffold 104 and inner chamber 105 with the desired porosity, other fabrication techniques may be used. For example, fabrication may be accomplished with any of the following: (1) solvent casting and particulate leaching, (2) gas foaming, (3) vacuum drying, (4) and thermally induced phase separation. These techniques offer a degree of control over the pore size and overall porosity of the components 104/105, although interconnectivity is not as well controlled. Despite this limitation, these alternative techniques are suitable as they permit fabrication of a scaffold 104 and inner chamber 105 with complementary shapes and porosity, while also ensuring that the pores are interconnected to facilitate perfusion.

Bioreactor System and Perfusion

The bioreactor system 100 and scaffold 104 can be used to culture a variety of tissues. The following example will discuss fabrication of bone substitute grafts. In this process, human mesenchymal stem cells derived from bone marrow (hMSCs) are passaged at 70% confluence and seeded for differentiation on top of Dental SG disks at a density of ˜1000 cells/mm² in mesenchymal stem cell culture media. Following 1 day of culture, cells are either maintained in mesenchymal stem cell culture media or switched into a commercial mesenchymal stem cell bone differentiation media (DM). Cells are differentiated for 14 days.

The sterile scaffold 104 and inner chambers 105 are immersed in 50 μg/mL collagen solution and anti-adherence rinsing solution, respectively. The parts are degassed for 3 hours while immersed in the respective solutions in a vacuum chamber under sterile conditions. Subsequently, both parts are dried in a biosafety cabinet to stabilize the coating. Next, both parts were immersed in Dulbecco's phosphate-buffered saline (DPBS) and degassed for 3 hours.

The scaffold 104 and inner chamber 105 are taken out of the DPBS solution and immediately assembled into the bioreactor 101 to prevent drying. The inlet 102 and outlet 103 of the bioreactor 100 are then joined. Once assembled, the bioreactor 101 is primed with DPBS. The assembly can be completed in a sterile manner in a biosafety cabinet.

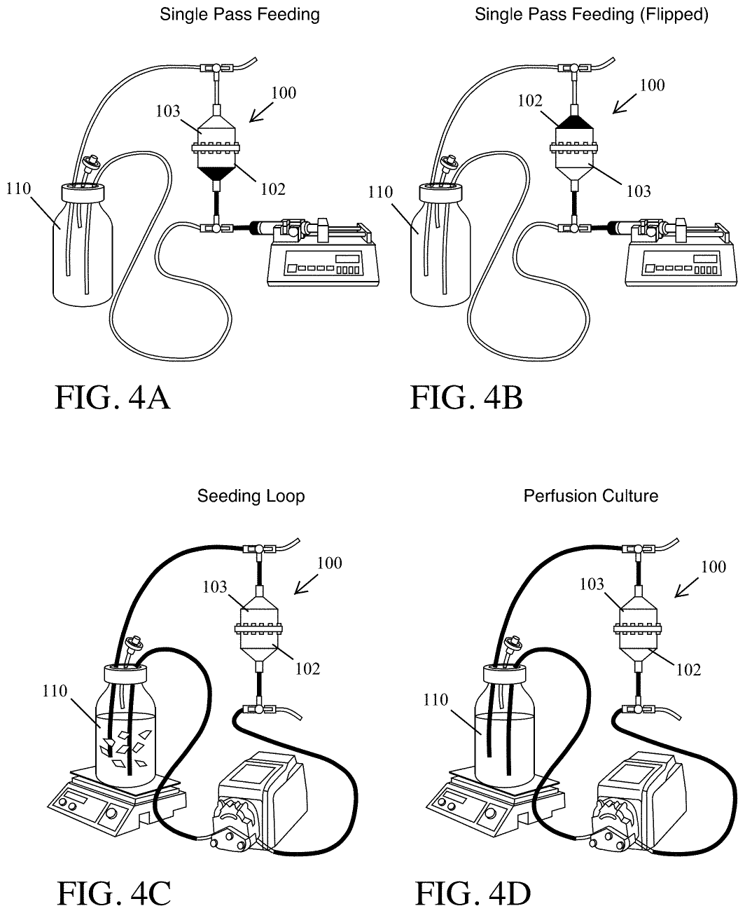

In this example, a single pass of 7.5 million hMSCs are perfused through the bioreactor 101 using a syringe pump at a rate of 10 mL/min (FIG. 4A). The bioreactor 101 is then flipped vertically and seeded with another pass of 7.5 million hMSCs (FIG. 4B). Unattached cells are collected in a media reservoir 110 and perfused in a loop for one hour using a peristaltic pump (FIG. 4C). The media reservoir 110 is stirred to avoid cell clumping. Additionally, the cells are left to rest without any perfusion for one hour. Following this, unattached cells are washed out of the media reservoir 110 using DPBS. Perfusion is then initiated with fresh media and a peristaltic pump at a rate of ˜1 mL/min (FIG. 4D). The scaffold 104 is harvested after one day of perfusion culture.

Results

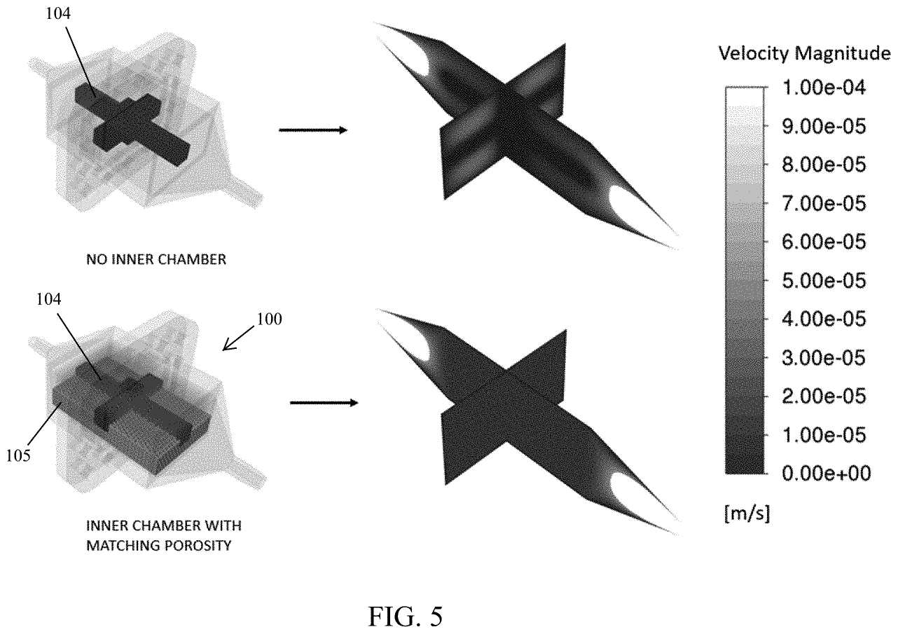

The effects of incorporating an inner chamber 105 that matches the porosity and geometry of the scaffold 104 can be first evaluated by performing a porous media simulation on ANSYS Fluent. FIG. 5 shows two separate simulations, one without an inner chamber (top image) and one with an inner chamber (bottom image). Different flow rates through the bioreactor 101 are shown according to the scale on the right of FIG. 5. Without an inner chamber 105, i.e., when the porosity in the inner chamber 105 is 100%, the fluid is most likely to flow through the void space surrounding the scaffold 104. That is, the greatest flow rate is in the areas surrounding the scaffold 104. This results in a non-uniform velocity field across the bioreactor 101, with barely any fluid flow through the scaffold 104 that provides a greater resistance to flow in this instance. In contrast, the presence of an inner chamber 105 with the same porosity as the scaffold 104 opposes the preferential flow of fluid, resulting in a uniform velocity distribution across the scaffold 104 through the bioreactor 101. The flow rates depicted in FIG. 5 show that the bioreactor 101 design emphasizes uniform flow and has the potential to facilitate homogenous bone formation.

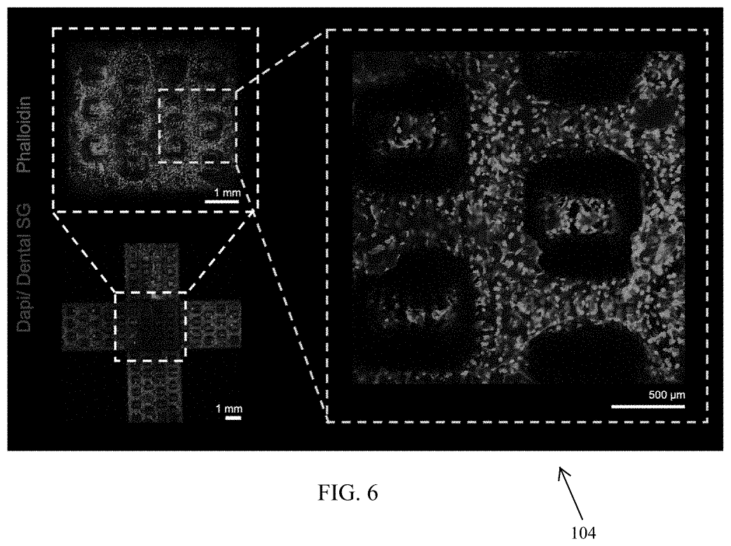

Additionally, the viability of a dynamic seeding of hMSCs in the bioreactor system 100 can be assessed. In one example assessment, the scaffold 104 may be stained with DAPI and phalloidin and to assess cell distribution using confocal microscopy (λ=405 nm, 455 nm respectively). FIG. 6 are confocal images confirming that hMSCs successfully adhered to the scaffold 104 under dynamic conditions with a relatively uniform distribution. Further, the cells attached at a fairly high confluence, a result beneficial for the initiation of differentiation into osteogenic lineage.

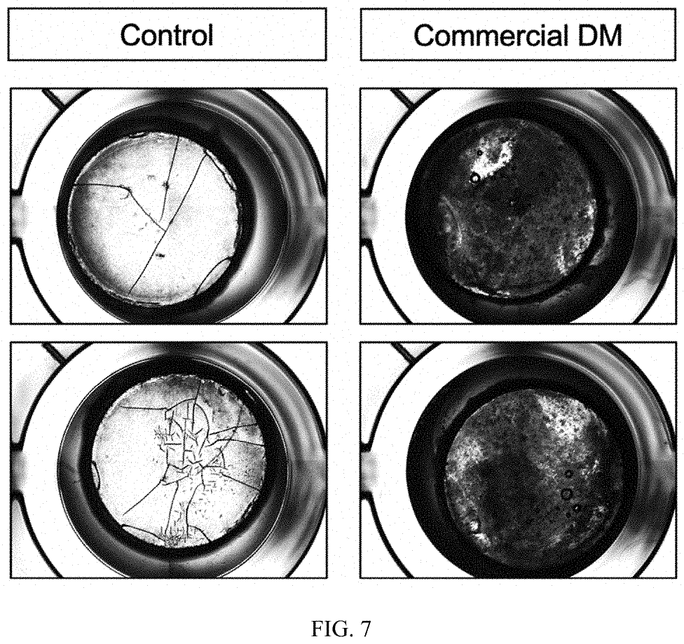

Static seeding and differentiation of hMSCs on a scaffold 104 can also be performed, as shown in FIG. 7. After 14 days, alizarin red staining confirmed that the scaffold 104 can support hMSC differentiation into osteogenic lineage (FIG. 7).

When used in this specification and claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

Protection may be sought for any features disclosed in any one or more published documents referenced herein in combination with the present disclosure.

Although certain example embodiments of the invention have been described, the scope of the appended claims is not intended to be limited solely to these embodiments. The claims are to be construed literally, purposively, and/or to encompass equivalents.

What is claimed is:

1. A bioreactor system comprising:
a bioreactor comprising an inlet and an outlet;
a scaffold disposed within an interior volume of the bioreactor,
wherein the scaffold has a plurality of interconnected pores defining a porosity and permitting a flow of fluid through the scaffold; and
an inner chamber positioned proximate to the scaffold,
wherein the inner chamber has a shape that complements a shape of the scaffold,
wherein a porosity of the inner chamber substantially matches the porosity of the scaffold;
wherein the scaffold and the inner chamber occupy the interior volume of the bioreactor.

2. The bioreactor system of claim 1, wherein the scaffold is fabricated using additive manufacturing techniques.

3. The bioreactor system of claim 1, wherein the scaffold comprises a plurality of struts arranged to form the plurality of pores.

4. The bioreactor system of claim 1, wherein the scaffold has a complex geometric shape.

5. The bioreactor system of claim 1, wherein the inner chamber has an inner cavity that matches an exterior shape of the scaffold.

6. The bioreactor system of claim 1, wherein the inner chamber comprises multiple pieces,
wherein the multiple pieces are adapted to fit a scaffold having a complex geometrical shape.

7. The bioreactor system of claim 1, wherein the porosity of the scaffold is in the range of 60% to 94%.

8. The bioreactor system of claim 1, wherein a fluid flows uniformly through the bioreactor.

9. The bioreactor system of claim 1, wherein a resistance to fluid flow of the scaffold is similar to a resistance to fluid flow of the inner chamber.

10. The bioreactor system of claim 1, wherein the scaffold is fabricated using one of the following techniques: solvent casting and particulate leaching, gas foaming, vacuum drying, and thermally induced phase separation.

11. The bioreactor system of claim 1, wherein the scaffold is coated with an extracellular matrix biomaterial and the inner chamber is coated with an anti-fouling agent.

12. A method of fabricating a bone graft comprising:
designing a scaffold based on a digital representation of a defect;
fabricating the scaffold using additive manufacturing techniques, wherein the scaffold has a porosity of about 60% or more;
fabricating an inner chamber having a porosity matching the porosity of the scaffold, wherein the inner chamber has an inner cavity matching an exterior shape of the scaffold;
placing the scaffold and inner chamber in a bioreactor comprising an inlet and outlet;
flowing a fluid through the bioreactor, wherein the fluid contains stem cells and stem cell culture media; and
culturing the stem cells attached to the scaffold.

13. The method of claim 12, further comprising:
rotating the bioreactor to reverse the orientation of the inlet and outlet; and
flowing an additional fluid through the outlet.

14. The method of claim 13, further comprising:
collected stem cells that do not attach to the scaffold in a media reservoir; and
perfusing the stem cells collected in the media reservoir through the bioreactor.

15. The method of claim 14, further comprising:
perfusing fresh culture media through the bioreactor.
